(19) United States
(12) Patent Application Publication
(10) Pub. No.: US 2007/0269390 A1
(43) Pub. Date: Nov. 22, 2007

(54) OIL-IN-WATER EMULSION

(75) Inventor: Mika Inoue, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/747,998

(22) Filed: May 14, 2007

Related U.S. Application Data
(60) Provisional application No. 60/802,505, filed on May 23, 2006.

(30) Foreign Application Priority Data
May 17, 2006 (FR) 06 04406

(51) Int. Cl. A61K 8/89; A61K 8/42

(52) U.S. Cl. 424/59; 424/70.22; 424/70.12; 977/926

(57) ABSTRACT

Oil-in-water emulsion containing (1) an emulsifying system containing at least one silicone surfactant of molecular weight greater than or equal to 10,000, at least one non-ionic cosurfactant, and at least one anionic cosurfactant, and (2) at least one monohydric alcohol containing from 2 to 6 carbon atoms, where the oils of the oily phase are soluble in the monohydric alcohol. These compositions exhibit good stability and good cosmetic properties. They can be used in many cosmetic and dermatological applications.

### REFERENCE TO PRIOR APPLICATIONS

This application claims priority to U.S. provisional application 60/802,505 filed May 23, 2006, and to French patent application 0604406 filed May 17, 2006, both incorporated herein by reference.

### FIELD OF THE INVENTION

The present invention relates to a composition for topical application in the form of an oil-in-water emulsion, to the process for preparing it, and to its use for example in treating, caring for, making up and/or cleansing the skin, the integuments (hair, eyelashes, nails) and/or the mucous membranes. The composition may in particular be a cosmetic and/or dermatological composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

For various reasons related in particular to better comfort during use (softness, emollience, and the like), the current cosmetic or dermatological compositions are mostly in the form of an emulsion of the oil-in-water (O/W) type, i.e. an emulsion consisting of a continuous dispersant aqueous phase and of a discontinuous dispersed oily phase.

Oil-in-water emulsions are generally stabilized with emulsifying surfactants suitable for O/W emulsions, which, by virtue of their amphiphilic structure, come to lie at the oily phase/aqueous phase interface and thus stabilize the droplets of oil dispersed in the aqueous phase. Despite the presence of emulsifiers, emulsions can have a tendency to phase-separate. For solving the problems of stability of conventional O/W emulsions, several solutions are known. For example, it is possible to increase the viscosity of the continuous phase (aqueous phase) with one or more water-soluble gelling agents. However, this solution is not suitable for obtaining fluid or very fluid products such as the products that constitute tonics and lotions, also called “cosmetic water”.

Another solution that can be envisaged for stabilizing fluid oil-in-water emulsions without the addition of thickeners or with a very low content of thickener, consists in producing “ultrafinell O/W emulsions in which the average size of the globules constituting the oily phase is within well-determined limits, i.e. between 50 and 1000 nm.

Several ultrafine emulsion techniques are known, for instance:
nanoemulsions, as described, for example, in documents EP-A-728460, EP-A-780114, EP-A-1010413, EP-A-1010414, EP-A-1010415, EP-A-1010416, EP-A-1013338, EP-A-1016453, EP-A-1018363, EP-A-1020219, EP-A-1025898 and EP-A-1172077. The drawback of this technology is the need to use mechanical energy and in particular a high-pressure homogenizer, which makes the process complicated;
PIT emulsions, as described, for example, in document EP-A-1297824, which do not require mechanical energy at a temperature in the phase inversion range. This process has the drawback of limiting the type of composition; thus, for example, it is difficult to modify the amounts of polyol and of alcohol since this leads to a modification of the phase inversion temperature. Another drawback of this technology is that the oily phase cannot be decreased to less than 50. Now, in the case of lotions and tonics, it may be desired to have O/W emulsions that contain a lot of water and less than 5% by weight of oils.

Alongside these techniques for preparing ultrafine emulsions, microemulsions which can also be fluid are known. Microemulsions, as described, for example, in document EP-A-0774482, are not strictly speaking emulsions, but are thermodynamically stable dispersions consisting of micelles of amphiphilic lipids swollen by oil, this oil generally comprising a very short chain (for example, hexane or decane) and being, moreover, solubilized by virtue of the joint presence of a large amount of surfactants and cosurfactants forming the micelles. The size of the swollen micelles is very small due to the small amount of oil that they can solubilize. This very small size of the micelles is the reason for their transparency. Microemulsions form spontaneously when the constituents (water, oils and surfactants) are simply brought into contact without any supply of mechanical energy other than simple magnetic stirring, regardless of the order of addition of the constituents. The major drawbacks of microemulsions are related to their high proportion of surfactants, leading to intolerances and resulting in a sticky feel when applied to the skin. Moreover, they generally have a very narrow range of formulation. These microemulsions are not therefore fine emulsions and cannot compensate for the drawbacks of the fine emulsions described above.

### SUMMARY OF THE INVENTION

There remains therefore the need for fine and fluid O/W emulsions which are stable, and processes that are less expensive and less complex than those of the prior art, i.e. including processes which do not require the provision of energy, regardless of whether this energy is mechanical or thermal, and therefore without involving high temperatures and without requiring material which provides a lot of energy, these processes being such that they have no effect on the chemical stability of the compounds constituting the composition.

The inventor has found, surprisingly, new fine and fluid O/W emulsions which are stable, and that it is possible to produce these fine emulsions without providing energy and without heating, through a specific choice of surfactants and of cosurfactants, and through the presence of alcohol.

The invention also relates to the uses of the emulsions, in particular in the cosmetics field, without modification or in the form of an article impregnated with said emulsions.

### DETAILED DESCRIPTION OF THE PREFERRED EMBODIMENTS

One subject of the present invention is a composition in the form of an oil-in-water emulsion, comprising an oily phase dispersed in an aqueous phase, wherein it comprises, consists essentially of, or consists of (1) an emulsifying system comprising at least one silicone surfactant of molecular weight greater than or equal to 10 000, at least one non-ionic cosurfactant, and at least one anionic cosurfactant, and (2) at least one monohydric alcohol containing from 2 to 6 carbon atoms, and in that the oil(s) of the oily phase is (are) soluble in the at least one monohydric alcohol.

The composition of the invention has the advantages of having good stability, even in the absence of thickeners in the aqueous phase and even at a very low oil content (for example 1 to 2%), of being easy to produce (conventional stirring at slow speed, without the need to heat, or to homogenize under high pressure), of allowing the amounts of alcohols and of polyols to be varied, of having a very good cosmetic quality, and of exhibiting good ocular and cutaneous tolerance, among other advantages.

The composition is preferably intended for topical application. The term “topical application” is here intended to mean an external application to keratin materials, namely, in particular, the skin, the scalp, the eyelashes, the eyebrows, the nails, the hair and/or the mucous membranes. Since the composition is intended for topical application, it preferably comprises a physiologically acceptable medium. The term “physiologically acceptable medium” is intended to mean a medium that is compatible with the skin, the lips, the scalp, the eyelashes, the eyes, the nails and/or the hair. In this regard the invention emulsion itself including its components can be the entire composition, which is preferably physiologically acceptable, the emulsion itself then being the physiologically acceptable medium. The composition may in particular constitute a cosmetic or dermatological composition.

The composition according to the invention is preferably stable at any pH. However, for topical application, it is preferable for the composition to have a pH ranging from 3 to 9, and better still from 3.5 to 8.5.

The invention composition preferably exhibits oily phase globules that have a number-average size (diameter) ranging from 50 to 1000 nm, more preferably from 100 nm to 500 nm, and even more particularly from 100 to 300 nm, this average size being an average size by intensity, measured by quasielastic light scattering, for example with the Model Instrument 90+ device from the company Brookhaven Instruments Corporation.

According to the size of the oily phase globules, the visual appearance of the composition according to the invention can range from a translucent or opalescent appearance to a white appearance. The fine emulsions of the invention preferably have a turbidity ranging from 100 to 900 NTU, and preferably from 200 to 600 NTU, the turbidity being measured with the HACH—Model 2100 P portable turbidimeter.

Emulsifying System

The composition according to the invention contains an emulsifying system containing at least one silicone surfactant of molecular weight greater than or equal to 10 000, at least one non-ionic cosurfactant, and at least one anionic cosurfactant.

The emulsifying system is present in an amount that can range, for example, from 0.07% to 5% by weight, preferably from 0.1% to 5% by weight, and better still from 0.2% to 3% by weight, relative to the total weight of the composition.

A. Silicone Surfactant

The term “silicone surfactant” is intended to mean a silicone compound comprising at least one oxyalkylenated chain, in particular comprising at least one oxyethylenated (—OCH₂CH₂—) and/or oxypropylenated (—OCH₂CH₂CH₂—) chain.

The silicone surfactant used in the present invention preferably should be soluble or dispersible in the monohydric alcohol and in particular in ethanol. It is also preferably chosen from surfactants that are liquid at ambient temperature.

Suitable silicone surfactants are those which have a molecular weight greater than or equal to 10 000, and they can be chosen in particular from, for example, polydimethylsiloxanes comprising both oxyethylenated groups and oxypropylenated groups. Mention may, for example, be made of the polydimethylsiloxane with an oxyethylene/oxypropylene ending sold as a mixture with caprylic/capric acid triglycerides under the name Abil care 85 by the company Goldschmidt (INCI name: BIS-PEG/PPG-16/16 PEG/PPG-16/16 Dimethicone/Caprylic/Capric Triglyceride), the polydimethylsiloxane comprising an alpha-omega polyether group (OE/OP: 40/60), sold under the name Abil B8832 by the company Goldschmidt (INCI name: BIS-PEG/PPG-20/20 Dimethicone), the oxyethylenated oxypropylenated polydimethylsiloxane sold under the name Abil B88184 by the company Goldschmidt (INCI name: PEG/PPG-20/6 Dimethicone) and the oxyethylenated oxypropylenated polydimethyl/methylsiloxane sold under the name Abil B8852 by the company Goldschmidt (INCI name: PEG/PPG-4/12 Dimethicone), and mixtures thereof.

According to a preferred embodiment of the invention, the silicone surfactant is Abil care 85.

The amount of silicone surfactant(s) preferably ranges from 0.05% to 4% by weight relative to the total weight of the composition.

According to a preferred embodiment of the invention, the weight ratio of the amount of oil(s) to the amount of silicone surfactant(s) ranges from 0.5 to 20, and better still from 1 to 10, and the weight ratio of the amounts of silicone surfactant(s) and of oil(s) to the amount of monohydric alcohol(s) ranges from 0.01 to 1, better still from 0.05 to 0.3, and even better still from 0.1 to 0.2.

In the present application, the term “amount of oils” is intended to mean the total amount of the oils present in the composition.

Moreover, according to a preferred embodiment of the invention, the weight ratio of the amount of anionic cosurfactant(s) to the amount of silicone surfactant(s) ranges from 0.01 to 1, and better still from 0.05 to 0.5.

B. Non-Ionic Cosurfactant

The composition according to the invention contains at least one non-ionic cosurfactant which is preferably chosen from non-ionic surfactants that have an HLB of less than or equal to 15.

The HLB (hydrophilic-lipophilic balance) of an emulsifying surfactant is calculated according to the following formula:

$$HLB = 20 \times \frac{m hydrophilic}{total m of T A}$$

in which m hydrophilic represents the weight of the hydrophilic group (i.e. the polar part) and total m of TA represents the total weight of the surfactant.

As non-ionic surfactants that may be used as cosurfactants in the present application, mention may in particular be made, for example, of polycondensates of ethylene oxide and of propylene oxide, and more particularly copolymers consisting of polyethylene glycol and polypropylene glycol blocks, for instance polyethylene glycol /polypropylene glycol /polyethylene glycol triblock polycondensates. These triblock polycondensates have, for example, the following chemical formula:
H—(O—CH₂—CH₂)ₐ—(O—CH(CH₃)—CH₂)_{b}—(O—CH₂—CH₂)ₐ—OH

where a ranges from 2 to 150, and b ranges from 1 to 100; preferably, a ranges from 10 to 130, and b ranges from 20 to 80.

The non-ionic cosurfactant preferably has a weight-average molecular weight ranging from 1000 to 15 000, better still ranging from 1500 to 15 000, in particular ranging from 1500 to 10 000, and even better still ranging from 1500 to 5000.

As polycondensates, mention may in particular be made, for example, of the polyethylene glycol/polypropylene glycol/polyethylene glycol triblock polycondensates sold under the names “Synperonic” by the company Uniqema, such as the ethylene oxide, propylene oxide and ethylene oxide condensate (13 OE/30 OP/13 OE) (MW: 2900) sold under the name Synperonic PE/L 64 (Poloxamer 184), the ethylene oxide, propylene oxide and ethylene oxide condensate (8 OE/30 OP/8 OE) (MW: 2500) sold under the name Synperonic PE/L 62 (INCI name: Poloxamer 182), the ethylene oxide, propylene oxide and ethylene oxide condensate (6 OE/67 OP/6 OE) (MW: 4400) sold under the name Synperonic PE/L 121 (INCI name: Poloxamer 401), the ethylene oxide, propylene oxide and ethylene oxide condensate (46 OE/16 OP/46 OE) (MW: 5000) sold under the name Synperonic® PE/F38 (INCI name: Poloxamer 108), the ethylene oxide, propylene oxide and ethylene oxide condensate (128 OE/54 OP/128 OE) (MW: 14 000) sold under the name Synperonic® PE/F108 (INCI name: Poloxamer 338), the ethylene oxide, propylene oxide and ethylene oxide condensate (11 OE/21 OP/11 OE) (MW: 2200) sold under the name Synperonic® PE/L44 (INCI name: Poloxamer 124), the ethylene oxide, propylene oxide and ethylene oxide condensate (5 OE/21 OP/5 OE) (MW: 1630) sold under the name Synperonic® PE/L42 (INCI name: Poloxamer 122), the ethylene oxide, propylene oxide and ethylene oxide condensate (98 OE/67 OP/98 OE) (MW: 12 000) sold under the name Synperonic® PE/F127 (INCI name: Poloxamer 407), the ethylene oxide, propylene oxide and ethylene oxide condensate (97 OE/39 OP/97 OE) (MW: 10 800) sold under the name Synperonic® PE/F88 (INCI name: Poloxamer 238), and mixtures thereof.

The ethylene oxide, propylene oxide and ethylene oxide condensate (75 OE/30 OP/75 OE) (MW: 8350) sold under the name Lutrol® F68 (INCI name: Poloxamer 188) by the company BASF, and the ethylene oxide, propylene oxide and ethylene oxide condensate (13 OE/30 OP/13 OE) (MW: 2900) sold under the name Pluracare L 64 by the company BASF (INCI name: Poloxamer 184) can also be used.

The non-ionic cosurfactant is preferably chosen from polyethylene glycol/polypropylene glycol/polyethylene glycol triblock polycondensates (Poloxamer) with an HLB of less than or equal to 15, in particular Poloxamer 184, Poloxamer 182 and Poloxamer 401, and mixtures thereof.

The amount of non-ionic cosurfactant(s) preferably ranges from 0.01-6 to 0.5% by weight relative to the total weight of the composition.

According to a preferred embodiment of the invention, the weight ratio of the amount of non-ionic cosurfactant(s) to the amount of oils ranges from 0.001 to 1, better still from 0.005 to 0.5, and even better still from 0.01 to 0.2.

C. Anionic Cosurfactants

The anionic cosurfactants that can be used in the context of the present invention are preferably chosen from the group comprising neutralized anionic lipids. They can in particular be chosen from lipoamino acids and salts thereof, such as mono- and disodium acylglutamates, for instance the disodium salt of N-stearoyl L-glutamic acid sold under the name Acylglutamate HS21P or Amisoft HS21P by the company Ajinomoto, the monosodium salt of N-lauroyl L-glutamic acid sold under the name Acylglutamate LS11 or Amisoft LS11 by the company Ajinomoto, and mixtures thereof.

The amount of anionic cosurfactant(s) preferably ranges from 0.01% to 0.5% by weight relative to the total weight of the composition.

Monohydric Alcohol

The composition according to the invention contains at least one monohydric alcohol containing from 2 to 6 carbon atoms. A monohydric alcohol is a primary alcohol that contains only one OH function.

The amount of monohydric alcohol(s) can range, for example, from 0.1% to 60%, and preferably from 5% to 30% of the total weight of the composition.

Preferably, the weight ratio of the amount of monohydric alcohol(s) to the amount of water ranges from 0.01 to 0.2, and the weight ratio of the amount of monohydric alcohol(s) to the amount of oil(s) ranges from 0.05 to 0.2.

As monohydric alcohols mention may, for example, be made of ethanol, propanol, butanol, isopropanol, isobutanol, and mixtures thereof. The monohydric alcohol is preferably ethanol.

Polyols

In addition to the monohydric alcohols, the composition may contain one or more polyols, for instance glycerol, polyglycerol or sorbitol, and glycols such as isoprene glycol, 1,3-butylene glycol, propylene glycol, dipropylene glycol and hexylene glycol, and mixtures thereof.

The amount of polyol(s) can range, for example, from 1% to 50%, preferably from 2% to 30%, and better still from 5% to 30% of the total weight of the composition.

Oily Phase

The oily phase is made up of the oils and all the lipophilic constituents that may be present in the composition of the invention.

The oily phase contains at least one oil. The term “oil” is intended to mean a fatty substance that is liquid at ambient temperature (25° C.).

The oils used in the composition of the invention are chosen from oils that are soluble or dispersible in the monohydric alcohol. The term “soluble” is intended to mean the fact that an amount of 5% is solubilized in the monohydric alcohol, i.e. that a solution at 5% by weight in the monohydric alcohol, and in particular in ethanol, is clear.

The oily phase preferably contains only oils that are soluble in the monohydric alcohol. However, it can also contain oily active agents that are insoluble in the monohydric alcohol in so far as these active agents are soluble in the oil and where, therefore, the combination oils+active agents can be solubilized in the monohydric alcohol, and in particular ethanol.

More specifically, the oil(s) that can be used in the composition of the invention is (are) preferably chosen either from oils that have a molecular weight of less than or equal to 350, or from oils that have a molecular weight of greater than 350 and have an IOB (inorganic/organic balance) value of greater than or equal to 0.1.

The IOB parameter is known to those skilled in the art from a certain number of publications, such as:
(1) “Prediction of organic compounds by a conceptional diagram”, A. FUJITA Pharm. Bull 2, 163-173 (1954)
(2) “Organic Analysis” Fujita (1930), published by Kaniya Shoten,
(3) “Prediction of Organic Compounds and Organic Conceptional Diagram” A. Fujita (Kagaku-no-Ryoiki 11-10)” (1957), pp. 719-725,
(4) “Systematic Organic Qualitative Analysis (Book of Purified Substances)” Fujita and Akatsuka (1970), p. 487, published by Kazama Shoten,
(5) “Organic Conceptional Diagram, Its Fundamentals and applications”, Koda (1984), p. 227, published by Sankyo Shuppan,
(6) “Design of Emulsion Formulations by use of Organic Conceptional Diagram” (1985), p. 98, Yaguchi, published by Nippon Emulsion K. K.,
(7) R. H. Ewell, J. M. Harrison, L. Berg.: Ind. Eng. Chem. 36, 871 (1944),
(8) EP-A-985404

The IOB of a compound corresponds to the ratio of the inorganic value of the compound to the organic value of the compound:

IOB=inorganic value/organic value.

To calculate the organic value of a compound, the methylene group is considered as unit and is evaluated by the number of carbon atoms. A carbon atom or a —CH₃, —CH₂—or ═CH— group is counted for a value of 20 (value without units). The hydrogen atoms are not taken into account. The presence of a ring, branching or an ethylenic or acetylenic unsaturation in said organic compound is taken into account in calculating the organic value of the compound according to the corresponding organic value known in the literature, in particular on page 167 of publication (1) mentioned above.

To calculate the inorganic value of a compound, the hydroxyl group is taken as the standard group, for which an inorganic value of 100 is attributed. This arbitrary value of 100 is correlated to the distance between the boiling point curve for the alkane series as a function of the number of carbon atoms in said alkane and the boiling point curve for the linear saturated primary monoalcohols analogous to the alkanes.

The inorganic value (noted as Ix) of a substituent X (i.e. of any atom other than carbon or hydrogen, and of any group of atoms other than the groups of atoms formed exclusively of carbon and/or hydrogen) is determined by means of graphs. This value Ix is calculated by determining, firstly, the boiling point (B.P.) of a linear alkane and the boiling point (B.P.ₓ) of the homologue of said linear alkane substituted with the substituent X, and then by calculating the difference ΔTx=B.P.ₓ−B.P., and, secondly, by determining the boiling point (B.P._{OH}) of the homologue substituted with a primary alcohol group, and then by calculating the difference ΔT_{OH}=B.P._{OH}−B.P. The value Ix is equal to the ratio of the difference ΔTₓ over ΔT_{OH}, said ratio all being multiplied by the inorganic value of the hydroxyl group, equal to 100.

$$Ix = \frac{Δ {T}_{x}}{Δ {T}_{OH}} \times 100$$

The inorganic values of many substituents are described in the literature, in particular in the references mentioned above. The inorganic value of a compound is calculated by adding the inorganic value of the (or of all of the) substituent(s) present in said compound.

Certain substituents have both an organic value and an inorganic value, as indicated in reference (1) mentioned above, page 167, such as, for example, the substituents —Cl or —F.

The IOB of a mixture of organic compounds is equal to the ratio of the sum of the inorganic values of said organic compounds in the mixture to the sum of the organic values of said organic compounds in the mixture.

As preferred oils that can be used in the composition of the invention, mention may, for example, be made of:
fatty acid esters, such as the oils of formula R¹COOR² in which R¹ represents the residue of a fatty acid containing from 8 to 29 carbon atoms, and R² represents a branched or unbranched hydrocarbon-based chain containing from 3 to 30 carbon atoms, for instance isononyl isononanoate (MW=284), isopropyl palmitate (MW=298), 2-ethylhexyl palmitate (MW=368); IOB=0.128), isopropyl isostearate (MW=326; IOB=0.15), caprylic/capric acid triglycerides (MW=494; IOB=0.314), pentaerythritol esters such as pentaerythrityl tetraoctanoate (INCI name: pentaerythrityl tetraethylhexanoate) (MW=640; IOB=0.353), di(2-ethylhexyl) adipate (MW=370; IOB=0.286) or C₁₂-C₁₅ alkyl benzoate (MW=309; IOB=0.184) (Finsolv TN);
fatty alcohols containing from 8 to 26 carbon atoms, in particular octyldodecanol (MW=300);
isohexadecane (MW=226), isododecane (MW=170);
silicone oils, in particular volatile silicone oils such as cyclopolydimethylsiloxanes (cyclomethicones), for instance cyclohexasiloxane and cyclopentasiloxane (MW=370; IOB=0.4), or phenyl silicones, for instance phenyl trimethicones (MW=372; IOB=0.212),
mixtures thereof.

According to a preferred embodiment of the invention, the oily phase contains one or more oils chosen from phenyl trimethicones such as the products sold under the names “Dow Corning 556 Cosmetic Grade Fluid” by the company Dow Corning, “Abil AV 1000” and “Abil AV 20 HS” by the company Goldschmidt, “KF 56” by the company Shin Etsu; pentaerythritol esters such as pentaerythrityl tetraoctanoate, for instance the products sold under the names “Nikkol Pentarate 408” by the company Nikko Chemicals, “Trivent PE-48” by the company Akzo, “Priolube 3929” by the company Uniqema, “Hatcol 5140” by the company Hatco; octyldodecanol; isohexadecane; isododecane; cyclopolydimethylsiloxanes; and mixtures thereof.

According to a particularly preferred embodiment, the oily phase contains at least one mixture of a pentaerythritol ester and of phenyl trimethicone, more particularly a mixture of pentaerythrityl tetraoctanoate and of phenyl trimethicone.

The oily phase can represent, for example, from 0.5% to 10% by weight, preferably from 0.5% to 8% by weight, and better still from 1% to 5% by weight, relative to the total weight of the composition.

Aqueous Phase

Conventionally, the dispersant aqueous phase comprises water and any of the water-soluble or water-dispersible adjuvants.

The composition according to the invention may comprise a considerable amount of water. This amount can range, for example, from 40% to 90% by weight, preferably from 50% to 85% by weight, and better still from 60% to 85% by weight, relative to the total weight of the composition.

Polymers

The composition can also contain one or more hydrophilic polymers.

The hydrophilic polymers, i.e. polymers that are soluble or dispersible in water, can be chosen, for example, from modified or unmodified carboxyvinyl polymers, such as the products sold under the names Carbopol (INCI name: carbomer) and Pemulen (INCI name: Acrylates/C₁₀-C₃₀ alkyl acrylate crosspolymer) by the company Noveon; polyacrylamides; optionally crosslinked and/or neutralized 2-acrylamido-2-methylpropanesulphonic acid polymers and copolymers, such as the poly-(2-acrylamido-2-methylpropanesulphonic acid) sold by the company Hoechst under the name “Hostacerin AMPS” (INCI name: ammonium polyacryldimethyltauramide); crosslinked anionic copolymers of acrylamide and of AMPS, in the form of a W/O emulsion, such as those sold under the name Sepigel 305 (CTFA name: Poly-acrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company Seppic; polysaccharide biopolymers, such as guar gum and derivatives such as hydroxypropylated guar gum, alginates, modified or unmodified celluloses such as methylhydroxyethylcellulose, hydroxypropylmethylcellulose or hydroxyethylcellulose; and mixtures thereof. These polymers can be present in an amount ranging, for example, from 0.001% to 26 by weight, preferably from 0.05% to 1.5% by weight, relative to the total weight of the composition.

According to a preferred embodiment of the invention, the polymers are chosen from Carbopol 980 (BF Goodrich), Carbopol ETD 2001 (BF Goodrich), Synthalen K (3V Sigma), Carbopol 981 (BF Goodrich), Synthalen L (3V Sigma), Carbopol 1382 (BF Goodrich), Pemulen TRl (BF Goodrich), Pemulen TR2 (BF Goodrich), Hostacerin AMPS (Clariant), more especially Carbopol 980 and Hostacerin AMPS.

Adjuvants

The composition according to the invention can contain any adjuvant, including those normally used in cosmetic or dermatological compositions.

Among the adjuvants that may be contained in the aqueous phase and/or in the oily phase of the emulsions in accordance with the invention, mention may in particular be made, for example, of antioxidants, emollients, cosmetic or dermatological active agents, fragrances, preserving agents, fillers, sequestering agents, pigments, dyes, or any other ingredient normally used in the fields under consideration.

Of course, those skilled in the art will take care to select the optional compound(s) to be added to the composition according to the invention, and the amounts thereof, in such a way that the advantageous properties intrinsically associated with the composition in accordance with the invention are not, or are not substantially, impaired by the addition envisaged.

As active agents, mention may, for example, be made of:
moisturizers such as, for example, sodium lactate; polyols, and in particular glycerol or sorbitol, polyethylene glycols; mannitol; amino acids; hyaluronic acid; lanolin; urea and mixtures containing urea, such as NMF (Natural Moisturizing Factor); liquid petroleum jelly; N-lauroylpyrrolidonecarboxylic acid and salts thereof; essential fatty acids; essential oils; and mixtures thereof;
anti-ageing active agents and keratolytic agents, such as α-hydroxy acids, and in particular acids derived from fruits, such as glycolic acid, lactic acid, malic acid, citric acid, tartaric acid or mandelic acid, derivatives thereof and mixtures thereof; β-hydroxy acids, for instance salicylic acid and derivatives thereof such as 5-n-octanoylsalicylic acid or 5-n-dodecanoylsalicylic acid; α-keto acids, such as ascorbic acid or vitamin C and its derivatives such as its salts, for instance sodium ascorbate, magnesium ascorbylphosphate or sodium ascorbylphosphate; its esters, for instance ascorbyl acetate, ascorbyl palmitate and ascorbyl propionate, or its sugars, for instance glycosylated ascorbic acid, and mixtures thereof; β-keto acids; retinoids such as retinol (vitamin A) and its esters, retinal, retinoic acid and its derivatives, and also the retinoids described in documents FR-A-2,570,377, EP-A-199636, EP-A-325540 and EP-A-402072; adapalene; carotenoids; and mixtures thereof;
vitamins, such as the vitamins A and C indicated above, and also such as vitamin E (tocopherol) and its derivatives; vitamin B3 (or vitamin PP or niacinamide) and its derivatives; vitamin B5 (or panthenol in its various forms: D-panthenol, DL-panthenol), and its derivatives and analogues, such as calcium pantothenate, pantethine, pantetheine, panthenyl ethyl ether, pangamic acid, pyridoxine, pantoyl lactose, and the natural compounds containing same, such as royal jelly; vitamin D and its analogues, such as those described in document WO-A-00/26167; vitamin F or its analogues, such as mixtures of unsaturated acids that have at least one double bond, and in particular mixtures of linoleic acid, linolenic acid and arachidonic acid, or the compounds containing same;
antibacterial and antiseborrheic agents, such as salicylic acid, 2,4,4′-trichloro-2′-hydroxydiphenyl ether (or triclosan), 3,4,4′-trichlorobanilide (or triclocarban), azelaic acid, benzoyl peroxide and zinc salts such as zinc lactate, zinc gluconate, zinc pidolate, zinc carboxylate, zinc salicylate and/or zinc cysteate.

The compositions of the invention can be used on any keratin materials such as the skin, the scalp, the hair, the eyelashes, the eyebrows, the nails or the mucous membranes. They can be used as skin care products, for example as protection, treatment or care creams for the face, for the hands or for the body, as body milks for protection or care of the skin, the scalp or the mucous membranes, or as hygiene products, for example as cleansing products for the skin or the mucous membranes, or else as hair products or as antisun products.

The compositions can also constitute products for making up the skin and/or the hair, for example incorporating pigments into the composition for constituting in particular foundations.

A subject of the invention is also the cosmetic use of the composition as defined above, as a skin care product, as a hygiene product, as a hair product, as an antisun product and as a makeup product.

Another subject of the invention is a process for the cosmetic treatment of a keratin material, such as the skin, the scalp, the hair, the eyelashes, the eyebrows, the nails and the mucous membranes, wherein a composition as defined above is applied to the keratin material.

Moreover, when the compositions according to the invention have the appropriate fluidity, they can also be used to impregnate water-insoluble substrates so as to constitute articles (such as wipes) for use in caring for, cleansing and/or removing makeup from the skin, the eyelashes and/or the lips. The water-insoluble substrate can comprise one or more layers and it can be chosen from the group comprising woven materials, nonwoven materials, foams, sponges, cotton wool, in sheets, balls or films. It can in particular be a nonwoven substrate based on fibres of natural origin (flax, wool, cotton, silk) or of synthetic origin (cellulose derivatives, viscose, polyvinyl derivatives, polyesters such as polyethylene terephthalate, polyolefins such as polyethylene or polypropylene, polyamides such as Nylon, acrylic derivatives). The nonwovens are described in general in Riedel “Nonwoven Bonding Methods & Materials”, Nonwoven World (1987). These substrates are obtained according to the usual processes of the technique for preparing nonwovens.

When the substrate is a nonwoven, a thick nonwoven, which does not roll up into a ball and which is solid enough not to disintegrate and not to peel when applied to the skin is preferably used. It should be absorbent, and soft, at least on one face for removing makeup from the eyes, in particular. As suitable nonwovens, mention may, for example, be made of those sold under the names Ultraloft 15285-01, Ultraloft 182-008, Ultraloft 182-010 and Ultraloft 182-016 by the company BBA, Vilmed M1519 Blau, Vilmed M 1550 N and 112-132-3 by the company Freudenberg, that sold under the name Norafin 11601-010B by the company Jacob Holm Industries, and the flocked nonwovens sold under the names Univel 109 and Univel 119 by the company Uni Flockage.

Moreover, this substrate may comprise one or more layers that have identical or different properties and may have elasticity and softness properties and other properties suitable for the desired use. The substrates may comprise, for example, two parts that have different elasticity properties, as described in document WO-A-99/13861, or comprise a single layer with different densities, as described in document WO-A-99/25318, or comprise two layers of different textures, as described in document WO-A-98/18441.

The compositions impregnated onto the substrate can contain any compound suitable for the desired purpose, for example foaming surfactants for obtaining cleansing wipes, or active care agents for obtaining skin care wipes. They can also be used for making up the skin, for example by impregnating the wipe with a composition containing pigments and which may constitute a foundation.

A subject of the invention is therefore also an article obtained by impregnating a water-insoluble substrate with a composition as defined above.

A subject of the invention is also the use of the composition as defined above, for preparing an article for use in caring for, removing makeup from, cleansing or making up the skin, the lips and/or the eyelashes.

The composition according to the invention can be obtained by means of any suitable process. According to a preferred embodiment of the invention, it is preferably prepared by means of the process comprising mixing, at ambient temperature (20 to 25° C.), the compounds of the aqueous phase except the monohydric alcohol, in preparing, at ambient temperature, the oily phase in the presence of the monohydric alcohol, and in pouring the oily phase into the aqueous phase with slow stirring. A very fine oil-in-water emulsion characterized by an oily globule size of less than 1000 nm, and preferably less than 300 nm, for example from 100 to 300 nm, and better still from 130 to 250 nm, and by an opalescent-to-white liquid appearance, is obtained.

A subject of the invention is thus also a process for preparing the composition as defined above, comprising:
mixing, at ambient temperature, the water, the non-ionic cosurfactant(s), the anionic cosurfactant(s) and the hydrophilic compounds except the monohydric alcohol,
mixing, at ambient temperature, the oils, the lipophilic active agents, the silicone surfactant(s) and the monohydric alcohol,
pouring the oily phase into the aqueous phase with slow stirring.

The following examples illustrate the invention without being limiting in nature. The amounts therein are percentages by weight. The compounds are indicated by their INCI name or by their chemical names, as appropriate.

Procedure:

In these examples, the process is carried out using the following procedure:

Phase A: The compounds of phase A were solubilized in water with stirring using a Rayneri mixer equipped with a deflocculating turbine, at ambient temperature.

Phase B: The compounds of phase B were solubilized in alcohol at ambient temperature.

Phase B was then poured into phase A with slow stirring.

### EXAMPLE 1

Phase A

| | |
|---|---|
| Water | 81.55% |
| Phenonip (mixture of preserving agents) | 0.5% |
| Glycerol | 5% |
| Dipropylene glycol | 3% |
| Sodium lauroyl glutamate | 0.03% |
| Disodium stearoyl glutamate | 0.02% |
| Poloxamer 184 | 0.1% |

Phase B

| | |
|---|---|
| Ethanol | 8% |
| Bis PEG/PPG-16/16 PEG/PPG-16/16 dimethicone | 0.4% |
| (and) caprylic/capric triglyceride | |
| (Abil Care 85) | |
| Pentaerythrityl tetraethylhexanoate | 1.6% |

A very fine oil-in-water emulsion characterized by an oily globule size of 220 nm, having the appearance of a white opalescent liquid, was obtained.

### EXAMPLE 2

### According to the Invention and Comparative Examples 1 to 4

| | | | |
|---|---|---|---|
| | | Comparative | Comparative |
| Composition | Example 2 | Example 1 | Example 2 |

Phase A

| | | | |
|---|---|---|---|
| Water | 81.85 | 81.85 | 81.85 |
| Dipropylene glycol | 3 | 3 | 3 |
| Glycerol | 3 | 3 | 3 |
| Disodium stearoyl | 0.05 | 0. 05 | 0.05 |
| glutamate | | | |
| Poloxamer 184 | 0.1 | 0.1 | 0.1 |
| Phenonip (preserving | 1 | 1 | 0.5 |
| agent) | | | |

Phase B

| | | | |
|---|---|---|---|
| 2-Ethylhexyl | 1 | 1 | |
| palmitate | | | |
| Caprylic/capric acid | | | 1 |
| triglycerides | | | |
| Bis-PEG/PPG-16/16 | 0.5 | | |
| dimethicone/caprylic/ | | | |
| capric triglyceride | | | |
| (Abil Care 85) | | | |
| PEG-20 glyceryl | | 0.5 | |
| triisostearate | | | |
| Sucrose myristate | | | 0.5 |
| Ethanol | 10 | 10 | 10 |
| Result of the | Stable | Release of | Release of |
| centrifugation (at | opalescent | oil 4 mm | oil 1 mm |
| 3000 rpm) | fluid | | |
| | emulsion | | |

| | |
|---|---|
| Comparative | Comparative |
| Example 3 | Example 4 |

Phase A

| | | |
|---|---|---|
| Water | 81.85 | 81.85 |
| Dipropylene glycol | 3 | 3 |
| Glycerol | 3 | 3 |
| Disodium stearoyl glutamate | 0.05 | 0.05 |
| Poloxamer 184 | 0.1 | 0.1 |
| Phenonip | 0.5 | 0.5 |

Phase B

| | | |
|---|---|---|
| Caprylic/capric acid | 1 | 1 |
| triglycerides | | |
| Polyglyceryl-10 isostearate | 0.5 | — |
| Lauroyl glycol hydroxypropyl | — | 0.5 |
| ether | | |
| Ethanol | 10 | 10 |
| Result of the centrifugation | Release | Release of |
| (at 3000 rpm) | of oil | oil and |
| | | phase |
| | | separation |

Comparative Examples 1 to 4 show that the use of surfactants other than the silicone surfactants claimed in the present application does not make it possible to achieve the objective of the invention and to obtain a stable fine emulsion.

### EXAMPLES 3 TO 5

### According to the Invention

| | | | |
|---|---|---|---|
| | Example 3 | Example 4 | Example 5 |
| | according | according | according |
| | to the | to the | to the |
| Composition | invention | invention | invention |

Phase A

| | | | |
|---|---|---|---|
| Water | 81.85 | 81.85 | 81.85 |
| Dipropylene glycol | 3 | 3 | 3 |
| Glycerol | 3 | 3 | 3 |
| Disodium stearoyl | 0.05 | 0.05 | 0.05 |
| glutamate | | | |
| Poloxamer 184 | 0.1 | 0.1 | 0.1 |
| Phenonip (preserving | 0.5 | 1 | 0.5 |
| agent) | | | |

Phase B

| | | | |
|---|---|---|---|
| 2-Ethylhexyl | | 1 | |
| palmitate | | | |
| Caprylic/capric acid | 1 | | 1 |
| triglycerides | | | |
| PEG/PPG-20/6 | 0.5 | | |
| dimethicone (Abil | | | |
| B88184) (MW 13 000) | | | |
| Bis-PEG/PPG-16/16 | | 0.5 | |
| dimethicone/caprylic/ | | | |
| capric triglyceride | | | |
| (Abil Care 85) | | | |
| (MW 11 000) | | | |
| Bis-PEG/PPG-20/20 | | | 0.5 |
| dimethicone (Abil | | | |
| B8832) (MW 10 000) | | | |
| Ethanol | 10 | 10 | 10 |
| Result of the | Stable | Stable | Stable |
| centrifugation (at | opalescent | opalescent | opalescent |
| 3000 rpm) | fluid | fluid | fluid |
| | emulsion | emulsion | emulsion |

### COMPARATIVE EXAMPLES 5 to 7

| | | | |
|---|---|---|---|
| | Comparative | Comparative | Comparative |
| Composition | Example 5 | Example 6 | Example 7 |

Phase A

| | | | |
|---|---|---|---|
| Water | 81.85 | 81.85 | 81.85 |
| Dipropylene glycol | 3 | 3 | 3 |
| Glycerol | 3 | 3 | 3 |
| Disodium stearoyl | 0.05 | 0.05 | 0.05 |
| glutamate | | | |
| Poloxamer 184 | 0.1 | 0.1 | 0.1 |
| Phenonip (preserving | 1 | 0.5 | 0.5 |
| agent) | | | |

Phase B

| | | | |
|---|---|---|---|
| Caprylic/capric acid | 1 | 1 | 1 |
| triglycerides | | | |
| PEG-14 dimethicone | 0.5 | | |
| (Abil B8843) (MW | | | |
| 5000) | | | |
| PEG/PPG-14/4 | | 0.5 | |
| dimethicone (Abil | | | |
| B8851) (MW 5000) | | | |
| PEG-12 dimethicone | | | 0.5 |
| (MW 3100) (Dow | | | |
| Corning 193 Fluid | | | |
| from Dow Corning) | | | |
| Ethanol | 10 | 10 | 10 |
| Result of the | Release of | Release of | Release of |
| centrifugation (at | oil | oil | oil |
| 3000 rpm) | (instability) | (instability) | (instability) |

Comparative Examples 5 to 7 show that the silicone surfactants of molecular weight less than 10 000 do not make it possible to achieve the object of the invention, i.e. to obtain a fine emulsion which is stable to centrifugation.

### EXAMPLES 6 AND 7

### According to the Invention and Comparative Examples 8 to 11

| | | | |
|---|---|---|---|
| | Example 6 | Example 7 | |
| | according | according | |
| | to the | to the | Comparative |
| Composition | invention | invention | Example 8 |

Phase A

| | | | |
|---|---|---|---|
| Water | 80.85 | 80.85 | 80.85 |
| Dipropylene glycol | 3 | 3 | 3 |
| Glycerol | 3 | 3 | 3 |
| Disodium stearoyl | 0.05 | 0.05 | 0.05 |
| glutamate | | | |
| Poloxamer 184 | 0.1 | 0.1 | 0.1 |
| Phenonip (preserving | 0.5 | 1 | 0.5 |
| agent) | | | |

Phase B

| | | | |
|---|---|---|---|
| Pentaerythrityl | 2 | | |
| tetraethylhexanoate | | | |
| Phenyl trimethicone | | 2 | |
| Hydrogenated | | | 2 |
| isoparaffin (MW = | | | |
| 350, IOB = 0) | | | |
| (insoluble in | | | |
| ethanol) | | | |
| Bis-PEG/PPG-16/16 | 0.5 | 0.5 | 0.5 |
| dimethicone/caprylic/ | | | |
| capric triglyceride | | | |
| (Abil care 85) (MW | | | |
| 11 000) | | | |
| Ethanol | 10 | 10 | 10 |
| Result | Stable | Stable | Impossible |
| | opalescent | opalescent | to produce |
| | fluid | fluid | the |
| | emulsion | emulsion | emulsion |

Comparative Examples 9 to 11 are analogous to Comparative Example 8, apart from the fact that the hydrogenated isoparaffin is replaced therein with other oils insoluble in ethanol, respectively with macadamia oil (MW greater than 500, IOB<0.1), isostearyl isostearate (MW=350 and IOB=0.086), pentaerythrityl tetraisostearate (MW640, IOB<0.1). In all cases, it was impossible to produce the emulsion.

### EXAMPLES 8 TO 10

### According to the Invention

| | Example 8 | Example 9 | Example 10 |
|---|---|---|---|
| | according | according | according |
| | to the | to the | to the |
| Composition | invention | invention | invention |
| Pentaerythrityl | 1 | 1 | 1 |
| tetraethylhexanoate | | | |
| Phenyl trimethicone | 1 | 1 | 1 |
| Bis-PEG/PPG-16/16 | 0.5 | 0.5 | 0.5 |
| dimethicone/caprylic/ | | | |
| capric triglyceride | | | |
| (Abil care 85) | | | |
| (MW 11 000) | | | |
| Ethanol | 10 | 10 | 10 |
| Water | 80.9 | 80.9 | 80.9 |
| Dipropylene glycol | 3 | 3 | 3 |
| Glycerol | 3 | 3 | 3 |
| Disodium stearoyl | 0.02 | 0.02 | 0.02 |
| glutamate | | | |
| Sodium N-lauryl | 0.03 | 0.03 | 0.03 |
| L-glutamate | | | |
| Poloxamer 401 | 0.2 | | |
| Poloxamer 182 | | 0.2 | |
| Poloxamer 184 | | | 0.2 |
| Phenonip (preserving | 0.5 | 0.5 | 0.5 |
| agent) | | | |
| Stability at 6 months | Good | Good | Good |

### EXAMPLE 11

### According to the Invention and Comparative Example 12

| | Example 11 | |
|---|---|---|
| | according to | Comparative |
| Composition | the invention | Example 12 |
| Pentaerythrityl | 1.6 | 1.6 |
| tetraethylhexanoate | | |
| Bis-PEG/PPG-16/16 | 0.34 | 0.34 |
| dimethicone/caprylic/capric | | |
| triglyceride (Abil care 85) | | |
| (MW 11 000) | | |
| Ethanol | 8 | 8 |
| Water | 81.35 | 81.35 |
| Dipropylene glycol | 3 | 3 |
| Glycerol | 5 | 5 |
| Disodium stearoyl glutamate | 0.02 | 0 |
| Sodium N-lauryl L-glutamate | 0.03 | 0 |
| Poloxamer 184 | 0.1 | 0.1 |
| Phenonip (preserving agent) | 0.5 | 0.5 |
| Appearance at time zero | Fine and stable | Phase |
| | emulsion | separation |

Comparative Example 12 shows that the presence of the anionic cosurfactant is essential in order to obtain a stable emulsion.

The above written description of the invention provides a manner and process of making and using it such that any person skilled in this art is enabled to make and use the same, this enablement being provided in particular for the subject matter of the appended claims, which make up a part of the original description and including a composition in the form of an oil-in-water emulsion, comprising an oily phase dispersed in an aqueous phase, wherein it contains (1) an emulsifying system containing at least one silicone surfactant of molecular weight greater than or equal to 10 000, at least one non-ionic cosurfactant and at least one anionic cosurfactant, and (2) at least one monohydric alcohol containing from 2 to 6 carbon atoms, and in that the oil(s) of the oily phase is (are) soluble in the monohydric alcohol.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition in the form of an oil-in-water emulsion having an oily phase comprising at least one oil dispersed in an aqueous phase comprising water, said emulsion comprising (1) an emulsifying system comprising at least one silicone surfactant of molecular weight greater than or equal to 10,000, at least one non-ionic cosurfactant, and at least one anionic cosurfactant, and (2) at least one monohydric alcohol having from 2 to 6 carbon atoms, wherein the oil(s) of the oily phase is (are) soluble in the at least one monohydric alcohol.

2. A composition according to claim 1, comprising at least one silicone surfactant chosen from polydimethylsiloxanes comprising oxyethylenated and/or oxypropylenated groups.

3. A composition according to claim 1, wherein the amount of silicone surfactant(s) is 0.05% to 4% by weight relative to the total weight of the composition.

4. A composition according to claim 1, comprising at least one non-ionic cosurfactant chosen from polycondensates of ethylene oxide and of propylene oxide.

5. A composition according to claim 1, comprising at least one non-ionic cosurfactant chosen from polyethylene glycol/polypropylene glycol/polyethylene glycol triblock polycondensates.

6. A composition according to claim 1, wherein the amount of non-ionic cosurfactant(s) is 0.01% to 0.5% by weight relative to the total weight of the composition.

7. A composition according to claim 1, comprising at least one anionic cosurfactant chosen from lipoamino acids and salts thereof.

8. A composition according to claim 1, wherein the amount of anionic surfactant(s) is 0.01% to 0.5% by weight relative to the total weight of the composition.

9. A composition according to claim 1, comprising ethanol.

10. A composition according to claim 1, wherein the amount of monohydric alcohol(s) is 0.1% to 60% by weight relative to the total weight of the composition.

11. A composition according to claim 1, wherein the weight ratio of the amount of oil(s) to the amount of silicone surfactant(s) is 0.5 to 20.

12. A composition according to claim 1, wherein the weight ratio of the amounts of silicone surfactant(s) and of oil(s) to the amount of monohydric alcohol(s) is 0.01 to 1.

13. A composition according to claim 1, wherein the weight ratio of the amount of monohydric alcohol(s) to the amount of water is 0.01 to 0.2.

14. A composition according to claim 1, wherein the weight ratio of the amount of anionic cosurfactant(s) to the amount of silicone surfactant(s) is 0.01 to 1.

15. A composition according to claim 1, further comprising one or more polyols.

16. A composition according to claim 1, comprising at least one oil chosen from oils that have a molecular weight of less than or equal to 350 and oils that have a molecular weight of greater than 350 and an IOB value of greater than or equal to 0.1.

17. A composition according to claim 1, comprising at least one oil chosen from phenyl trimethicones, pentaerythritol esters, octyldodecanol, isohexadecane, isododecane, cyclopolydimethylsiloxanes, and mixtures thereof.

18. A composition according to claim 1, wherein the amount of oily phase is 0.5% to 10% by weight relative to the total weight of the composition.

19. A composition according to claim 1, wherein the oily phase is dispersed in globules whose average size is 50 to 1000 nm.

20. A composition according to claim 1, wherein said composition is a skin care product, a hygiene product, a hair product, an antisun product or a makeup product.

21. A process comprising applying a composition according to claim 1 to keratin material.

22. An article comprising a water-insoluble substrate impregnated with a composition according to claim 1.

23. A process for preparing a composition in the form of an oil-in-water emulsion having an oily phase comprising at least one oil dispersed in an aqueous phase comprising water, said emulsion comprising (1) an emulsifying system comprising at least one silicone surfactant of molecular weight greater than or equal to 10,000, at least one non-ionic cosurfactant, and at least one anionic cosurfactant, and (2) at least one monohydric alcohol having from 2 to 6 carbon atoms, wherein the oil(s) of the oily phase is (are) soluble in the at least one monohydric alcohol,
said process comprising:
mixing, at ambient temperature, water, the non-ionic cosurfactant(s), the anionic cosurfactant(s) and any hydrophilic compounds except the monohydric alcohol(s),
mixing, at ambient temperature, the oil(s), any lipophilic active agents, the silicone surfactant(s) and the monohydric alcohol,
pouring the oily phase into the aqueous phase with stirring.
